(19) United States
(12) Patent Application Publication
(10) Pub. No.: US 2006/0081265 A1
(43) Pub. Date: Apr. 20, 2006

(54) Male urinary incontinence control device

(76) Inventor: Steven S. Warden, Virginia Beach, VA (US)

Correspondence Address: WILLIAMS MULLEN, FOUNTAIN PLAZA THREE, SUITE 200, 721 LAKEFRONT COMMONS, NEWPORT NEWS, VA, 23606, US

(21) Appl. No.: 11/252,267

(22) Filed: Oct. 17, 2005

Related U.S. Application Data
(60) Provisional application No. 60/619,255, filed on Oct. 15, 2004.

(51) Int. Cl. A61F 5/48

(52) U.S. Cl. 128/885

(57) ABSTRACT

A device for treating male urinary incontinence includes multiple pairs of interlocking locking rings formed with sections of latex which is twisted when the locking rings are mated. The device slips over the glans of the penis for positioning along the longitudinal axis of the penile shaft. When the locking rings are mated, pressure is applied to several points along the length of the urethra such that the urethral passageway is closed thus preventing flow therethrough. Release of the locking rings allows flow through the urethra.

### CROSS REFERENCE TO RELATED APPLICATIONS

The present application claims priority from U.S. Provisional Application Ser. No. 60/619,255, filed Oct. 15, 2004.

### BACKGROUND OF THE INVENTION

1. Field of the Invention

The invention relates to a device for controlling incontinence in males and more particularly to an incontinence device which controls incontinence with multiple pressure points and minimal discomfort.

2. Description of Related Art

Male urinary incontinence occurs when bladder pressure exceeds urethral closing pressure. This type of incontinence occurs spontaneously or more commonly after disease interventions that disrupt the normal control mechanisms.

Male urinary incontinence occurs after radical prostatectomy treatment for prostate cancer. It also occurs after neurological diseases that disrupt the neuronal controls to the urinary bladder and to the muscular sphincters, which control urinary leakage.

Normal physiological enlargement of the prostate can presage bladder instability resulting in an overactive bladder and leakage. Neurological disease states such as multiple sclerosis, strokes, spinal cord injuries, neoplasm, and peripheral nerve ailments can also end in urinary incontinence.

Urinary incontinence devices have attempted to solve these problems by obstructing the outflow of urine using penile compression and or urethral compression devices. Numerous devices precede the current yet are different in their mechanism of action securing urethral closure.

U.S. Pat. No. 6,904,916 discloses a male incontinence treatment device which consists of a belt which provides pressure against the urethra to prevent male incontinence. U.S. Pat. No. 6,843,253 discloses a urinary control device which is a hinged clamp. The inner surface of the clamp includes a protrusion which collapses the urethra preventing flow therethrough. U.S. Pat. No. 6,827,085 discloses a male urinary incontinence control device which is a strap. Pressure applying bolsters are threaded onto the strap at specific location such that pressure is applied to the urethra and flow therethrough is limited or prevented.

These types of devices act to control incontinence by tightening around the penis to prevent or significantly limit the flow of urine through the urethra. The pressure required by such devices is significant and causes a significant amount of discomfort. Additionally, much of the pressure applied is not directed at the urethra and thus, causes unnecessary pain. These devices must be removed periodically throughout the day because they restrict blood flow.

U.S. Pat. No. 6,805,662 discloses a urinary incontinence control device which uses an inflatable cuff to apply pressure to the urethra to prevent flow therethrough.

An object of the present invention is to provide an incontinence device which controls urinary incontinence in males.

An object of the present invention is to provide an incontinence device which controls urinary incontinence in males with minimal pressure.

An object of the present invention is to provide an incontinence device which controls urinary incontinence in males with minimal discomfort.

Finally, it is an object of the present invention to accomplish the foregoing objectives in a simple and cost effective manner.

### SUMMARY OF THE INVENTION

The present invention addresses these needs by providing a male urinary incontinence device in the form of a cuff which is worn on the shaft of a penis. The device includes a plurality of locking rings, preferably three to five rings which have an inner circumference and an outer circumference. The locking rings are preferably made from a substantially rigid, fluid impermeable material such as plastic or latex and are then preferably dipped in a fluid impermeable material such as latex to form flexible sections in between the rings. The locking rings are designed to mate to adjacent rings in a releasable fashion. In the preferred embodiment, the rings include teeth which mate with teeth on adjacent locking rings. As the rings are mated, the flexible material is twisted to exert compressive force on the shaft of the penis. Preferably, each portion of flexible material is twisted in the direction opposite to the direction the adjacent portions of flexible material is twisted. Another embodiment of the present invention includes forming a membrane across the inner circumference of at least one of the locking rings. This membrane includes a formed opening or the opening may be created by forming a cut across the center line of the membrane. Another embodiment of the present invention includes a plurality of elongate members extending from the inner circumference of the cuff parallel to the longitudinal axis of the cuff. The elongate members are preferably formed from the same material as the locking rings.

In use, the flexible cuff is installed on the shaft of a penis, such that the cuff circumscribes the penis. Each adjacent pair of rings is then mated by twisting the flexible cuff in between the locking rings. Preferably, the twisting motion is performed in opposite directions as each adjacent set of rings is mated. The locking rings are mated, preferably through the use of interlocking teeth which maintain the cuff in the twisted orientation until the teeth are released. Each twisted portion of the cuff provides an independent compressive force against the shaft of the penis.

### BRIEF DESCRIPTION OF THE DRAWINGS

A more complete description of the subject matter of the present invention and the advantages thereof, can be achieved by the reference to the following detailed description by which reference is made to the accompanying drawings in which:

FIG. 1 is a front view of an incontinence device according to the present invention;

FIG. 2 is a side view of the incontinence device according to the present invention in the untwisted orientation;

FIG. 3a is a perspective view of the preferred embodiment of the present invention in the untwisted orientation;

FIG. 3b is a perspective view of the preferred embodiment of the present invention in the twisted orientation;

FIG. 4 is a cross sectional perspective view of the preferred embodiment of the present invention in the untwisted orientation;

FIG. 5a is cross sectional view of the preferred embodiment of the present invention in use, in the untwisted orientation;

FIG. 5b is cross sectional view of the preferred embodiment of the present invention in use, in the twisted orientation;

FIG. 6 is a front view of an alternate embodiment of the present invention;

FIG. 7 is a side view of an alternate embodiment of the present invention;

FIG. 8 is a top view of an alternate embodiment of the present invention;

FIG. 9 is a perspective view of an alternate embodiment of the present invention; and

FIG. 10 is a front view of another alternate embodiment of the present invention.

### ELEMENT LIST

20 incontinence device
22 locking ring
24 teeth
26 latex tubing
28 urethra
30 extension
32 membrane

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention.

This male urinary incontinence device uses multiple urethral pressure points to achieve continence control. The device preferably uses a twisted condom of latex with locking mechanisms to assure urinary dryness. This twisted diaphragm principle using multiple areas of urethral compression is unique for male urinary incontinence. The reliance on multiple pressure points avoids the excessive pressures of other devices that render them undesirable. Each pressure point adds to the total pressure effect assuring dryness without over compressing the penis or the urethra. This in turn assures more comfort and reliability over previous devices.

The male urinary incontinence device for urethral compression utilizes the closure effect of twisted latex tubing as a diaphragm to gently apply pressure to the male urethra. Replicating these pressure phenomena at multiple adjacent sites creates a series effect reducing the individual pressures needed to secure urinary continence.

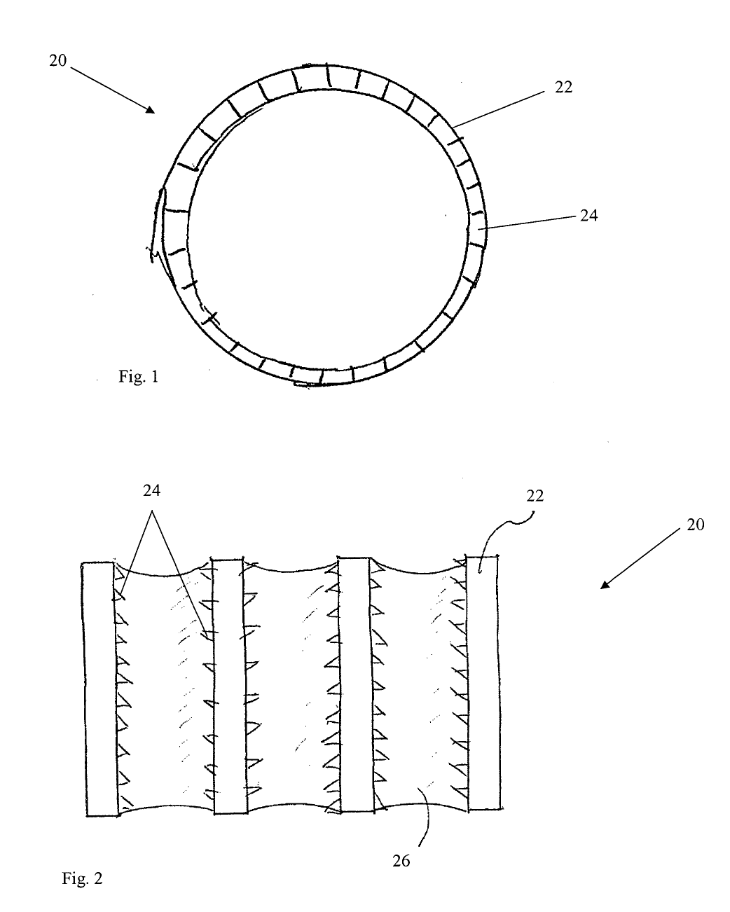

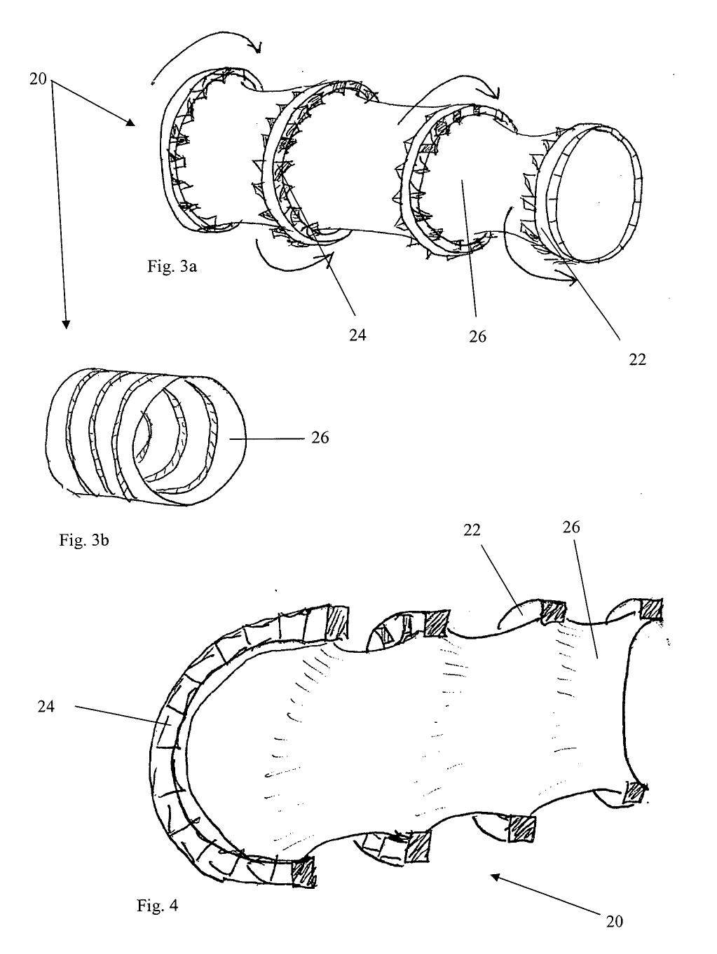

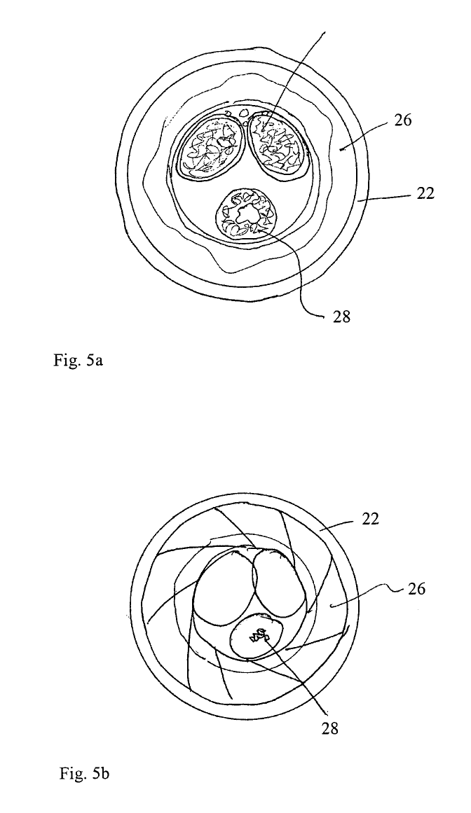

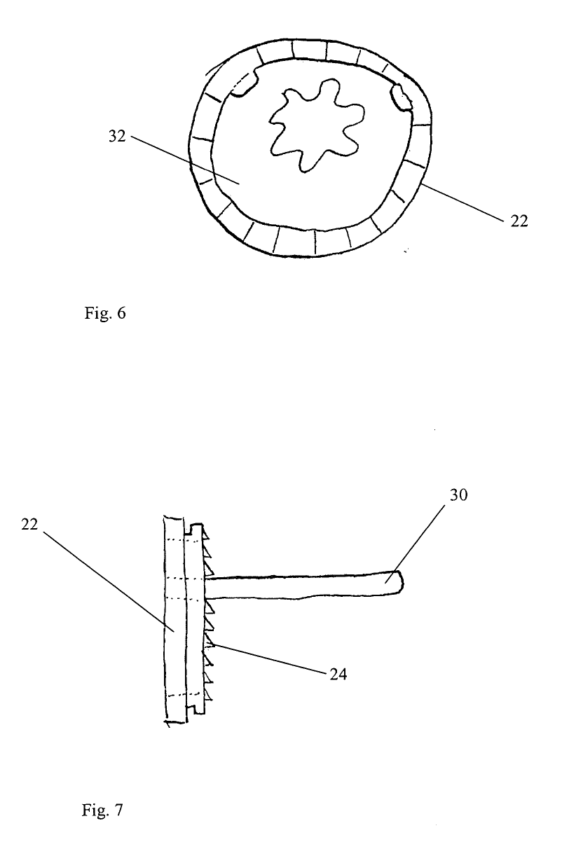

The incontinence device according to the present invention is shown in FIGS. 1-5. The incontinence device 20 is preferably made in a fashion similar to a latex condom using a dipping technique. While latex is the preferred material for the tubing portion 26 of the device 20, other material which would be appropriate may be used. The locking rings 22 are preferably latex or plastic or a similar material and are formed before dipping in latex to form the tubing portion 26. The locking rings 22 preferably have opposite directional teeth 24 that mate with teeth 24 on an adjacent locking ring 22 to prevent untwisting. The locking rings 22 are twisted in alternating directions causing the latex tubing 26 in between each set of locking rings 22 to twist in opposite directions as well. The bias of the latex tubing 26 toward untwisting maintains the locking rings in the twisted configuration. The latex tubing 26 can be untwisted by gently separating the locking rings 22 thus allowing the latex tubing 26 to untwist. The device 20 preferably has three, four or five locking rings 22.

As the latex tubing 26 is twisted it compresses an isolated area of the male urethra 28. The latex tubing 26 adjacent to this area is twisted the opposite direction compressing the second isolated area of male urethra 28. Continence is achieved by the multiple urethral closure pressures achieved at each of these isolated sites. The number of sites depends on the leak point pressure of the detrusor muscle precisely when urinary leakage occurs and on the available length of male urethra 28 for compression. The device 20 compresses the male penis secured behind the glands penis situated behind the corona of the head of the penis.

The device can also be used to secure external urinary collecting devices to the penis. The typical device is called a condom catheter or a “Texas catheter” and is secured with tape or adhesive materials. This male continence device 20 can be used to keep such collecting devices in place without the skin problems commonly seen with the alternative attachment mechanisms.

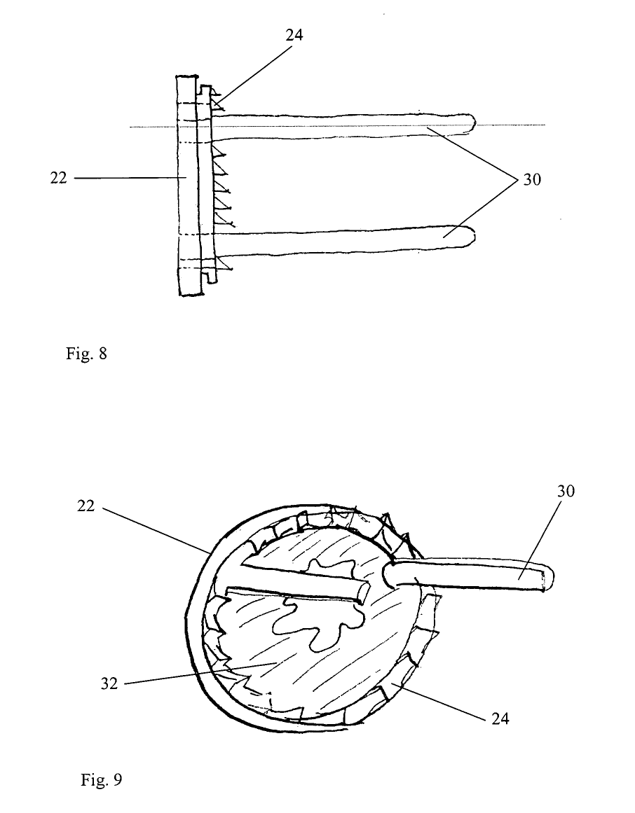

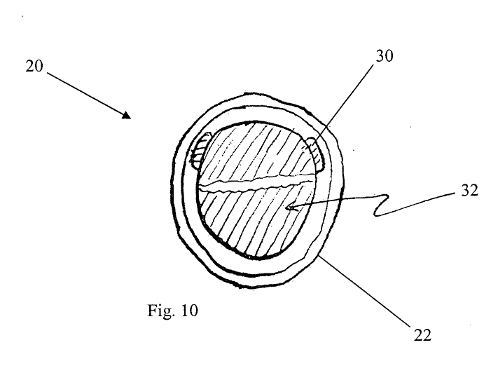

FIGS. 6-10 show an alternate embodiment of the present invention. In this embodiment, at least one of the locking rings 22 includes a membrane 32 across the opening in the center of the locking ring 22. The membrane 32 preferably includes an opening as shown in FIG. 6 or may include a cut in the surface of the membrane 32 as shown in FIG. 10. As shown in FIGS. 7-9, the locking ring 22 further includes a plurality of extensions 30 which extend from the inner circumference of the locking ring 22 such that the extensions 30 do not interfere with the teeth 24 along the circumference of the locking ring 22. The extensions are generally parallel to the urethra 28 when the device 20 is in use.

Many improvements, modifications, and additions will be apparent to the skilled artisan without departing from the spirit and scope of the present invention as described herein and defined in the following claims.

What is claimed is:

1. A male urinary incontinence device, comprising:
a plurality of locking rings, each locking ring having an inner circumference and an outer circumference;
a flexible material disposed between the plurality of mating locking rings such that a hollow cuff is formed which is adapted to encircle a shaft of a penis; and
a mating means for releasably mating the locking rings such that when the locking rings are mated, the flexible material is twisted to exert compressive force on the shaft of the penis.

2. The male urinary incontinence device as set forth in claim 1 wherein the plurality of locking rings are made from a substantially rigid, fluid impermeable material.

3. The male urinary incontinence device as set forth in claim 1 wherein the plurality of locking rings are made from a material selected from the group consisting of plastic and latex.

4. The method for treating male incontinence as set forth in claim 1 wherein the plurality of locking rings comprises at least three locking rings.

5. The method for treating male incontinence as set forth in claim 4 wherein the flexible cuff is twisted in opposite directions in between each adjacent pair of locking rings.

6. The male urinary incontinence device as set forth in claim 1 wherein the number of locking rings is selected from the group consisting of three, four and five.

7. The male urinary incontinence device as set forth in claim 1 wherein the flexible material is made from a fluid impermeable material.

8. The male urinary incontinence device as set forth in claim 1 wherein the flexible material is latex.

9. The male urinary incontinence device as set forth in claim 1 wherein the flexible material is disposed between the plurality of mating locking rings through a dipping process.

10. The male urinary incontinence device as set forth in claim 1 wherein the mating means is a plurality of teeth formed around the circumference of the locking ring which mate with a plurality of teeth formed around the circumference of an adjacent locking ring.

11. The male urinary incontinence device as set forth in claim 1 further comprising at least one of the plurality of locking rings having a membrane across the inner circumference of the locking ring wherein the membrane includes an opening.

12. The male urinary incontinence device as set forth in claim 1 further comprising at least one of the plurality of locking rings having at least one elongate member extending from the inner circumference of the locking ring such that the elongate member is substantial parallel to the longitudinal axis of the cuff.

13. A method for treating male incontinence, comprising:
providing a flexible cuff having a plurality of locking rings along the length of the cuff;
installing the flexible cuff on the shaft of a penis, such that the cuff circumscribes the penis; and
mating the locking rings with a twisting motion such that the flexible cuff is twisted between each adjacent pair of locking rings to provide a compressive force against the shaft of the penis.

14. The method for treating male incontinence as set forth in claim 13 wherein the plurality of locking rings comprises at least three locking rings.

15. The method for treating male incontinence as set forth in claim 14 wherein the flexible cuff is twisted in opposite directions in between each adjacent pair of locking rings.

16. The method for treating male incontinence as set forth in claim 13 wherein the locking rings are mated by means of a plurality of teeth formed along the circumference of each locking ring and along the surface of the locking ring such that the plurality of teeth can mate with a plurality of teeth formed on an adjacent locking ring.
